(19) United States
(12) Patent Application Publication
(10) Pub. No.: US 2005/0074130 A1
(43) Pub. Date: Apr. 7, 2005

(54) Stethoscope apparatus

(76) Inventors: Mark Brummel, Zeeland, MI (US); Phillip March, Holland, MI (US)

Correspondence Address: King & Jovanovic, PLC, 170 College Avenue, SUITE 230, HOLLAND, MI, 49423, US

(21) Appl. No.: 10/865,234

(22) Filed: Jun. 10, 2004

Related U.S. Application Data
(60) Provisional application No. 60/477,276, filed on Jun. 10, 2003.

(51) Int. Cl. A61B 7/04; A61B 5/02

(52) U.S. Cl. 381/67; 600/528

(57) ABSTRACT

A stethoscope apparatus comprising a body, a sensor assembly and an output indicator device. The body includes a body interfacing surface. The sensor assembly includes a sensor associated with the body interfacing surface. The output indicator device is coupled with the sensor assembly so as to provide at least one of a visual, audio and tactile indicators of the condition of the sensor. A storage device may be included to store data pertaining to the sensor. Similarly, a remote communication assembly may be incorporated to provide remote communication with the sensor assembly.

### CROSS-REFERENCE TO RELATED APPLICATION

This application claims the benefit of U.S. Provisional Patent Application Ser. No. 60/477,276 filed Jun. 10, 2003, the entire specification of which is incorporated herein.

### BACKGROUND OF THE INVENTION

1. Field of the Invention

The present invention relates in general to a stethoscope, and more particularly, to a stethoscope apparatus which provides numerous audio/visual, handling and data recording advantages.

2. Background Art

For years doctors have been utilizing conventional stethoscopes. Such stethoscopes include a pair of tubes connected to ear engaging members at one end and engaged with a diaphragm at the other end. The sound waves captured by the movement of the diaphragm are directed through the tubes to the ear of the wearer. As such the wearer is able to hear the beating heart of the patient.

Such stethoscopes, while greatly useful and in widespread use, suffer from certain drawbacks. First, such stethoscopes are not conducive for use by individuals that are hearing impaired. Moreover, they are difficult to utilize in noisy environments (i.e., accident scenes, etc.). Furthermore, it is often difficult to utilize conventional stethoscopes as the tubes become tangled in debris, become contaminated (i.e., water, sand, etc.), or become the object of a child's entertainment (during treatment). Additionally, as the tubes are of a generally uniform length, it is often difficult to reach certain areas, and use of the device requires one hand of the user, making it difficult to operate other equipment or to render necessary assistance without the help of another individual.

Accordingly, it is an object of the invention to provide for a stethoscope apparatus that overcomes the deficiencies of the prior art.

This an other objects will become apparent in light of the specification and claims appended hereto.

### SUMMARY OF THE INVENTION

A stethoscope apparatus comprising a body, a sensor and an output. The body includes a body interfacing surface. The sensor assembly includes a a sensor associated with the body interfacing surface. The output indicator device is coupled with the sensor assembly so as to provide at least one of a visual, audio and tactile indicators of the condition of the sensor.

In a preferred embodiment, the invention further comprises a storage device capable of storing data pertaining to the sensor.

In another preferred embodiment, the invention further comprises a remote communication assembly facilitating the transfer of data or sensor condition to a remote receiver or device.

### BRIEF DESCRIPTION OF THE DRAWINGS

The invention will now be described with reference to the drawings wherein:

FIG. 1 of the drawings comprises a perspective view of the stethoscope apparatus of the present invention;

FIG. 2 of the drawings comprises an embodiment of the stethoscope apparatus, showing, in particular, one grasping assembly thereof.

FIG. 3 of the drawings comprises an embodiment of the stethoscope apparatus showing, in particular, another grasping assembly thereof.

### DETAILED DESCRIPTION OF THE INVENTION

While this invention is susceptible of embodiment in many different forms, there is shown in the drawings several specific embodiments with the understanding that the present disclosure is to be considered as an exemplification of the principles of the invention and is not intended to limit the invention to the embodiments illustrated.

It will be understood that like or analogous elements and/or components, referred to herein, are identified throughout the drawings by like reference characters. In addition, it will be understood that the drawings are merely representations of the present invention, and some of the components may have been distorted from actual scale for purposes of pictorial clarity.

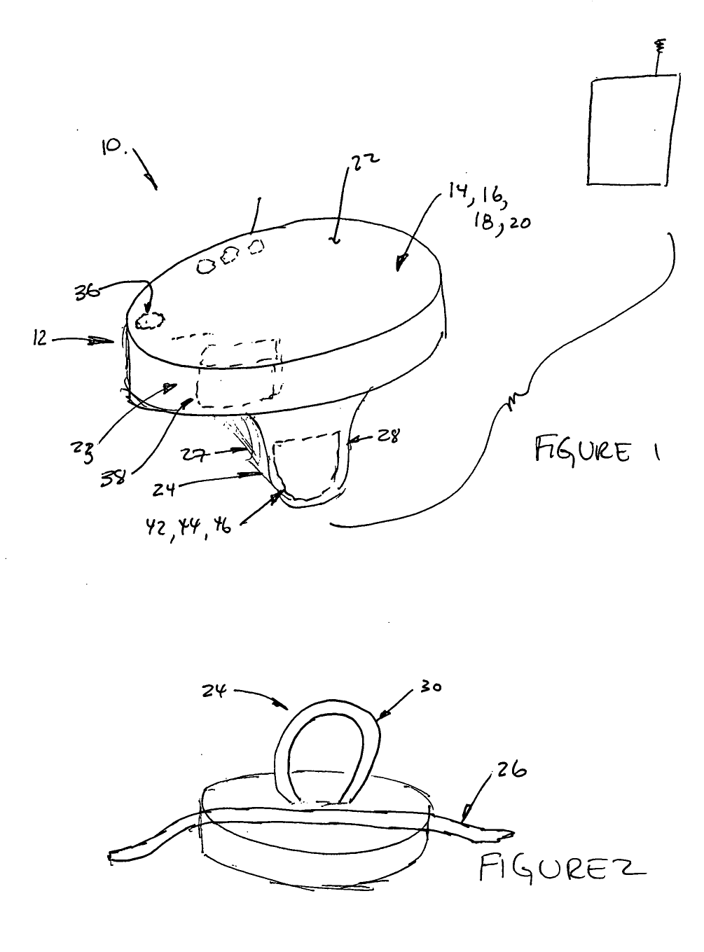

Referring now to the Figures, and in particular to FIG. 1, stethoscope apparatus 10 is shown as comprising body 12, sensor assembly 14, output indicator devices 16, storage device 18 and remote communication assembly 20. It will be understood that the stethoscope is suitable for use in association with both animals and humans. Moreover, it will be understood that the stethoscope apparatus may be configured for use in any number of environments (i.e., water-resistant, water-proof, dust-proof, etc.).

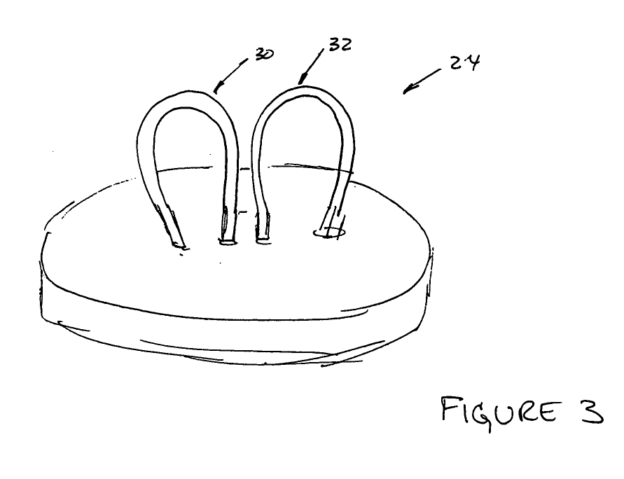

Body 12 is shown in FIG. 1 as comprising body interfacing surface 22, inner cavity 23, grasping assembly 24 and body adhesion assembly 26 (FIG. 2). Body interfacing surface 22 comprises a substantially thin surface which is capable of intimate abutment with the outer surface of a patient (i.e., the patient's skin). For example, such surfaces generally comprise a resiliently flexible polymer material which is substantially planar and generally rather thin. Such a material is capable of readily transferring sound waves therethrough. In certain embodiments, the body interfacing surface may be supplemented with a heating element which heats the body interfacing surface prior to use thereof. The heating element may be activated by a button, or other tactile device.

Inner cavity 23 is associated with interfacing surface 22 and is sized so as to readily accept sensor assembly 14, output indicator devices 16 and storage devices 18 therewithin, as needed. It is contemplated that the cavity may be defined by a plurality of wall structures which may be formed from metals, polymers, composites and combinations thereof. For example, the cavity may be defined by an outer structure which comprises a molded polymer material. It is likewise contemplated that the outer structure may comprise a partially molded polymer having metal member associated therewith. Of course, the invention is not limited to any particular material or any particular configuration of the inner cavity.

Grasping assembly 24 is shown in FIG. 1 as comprising opposing clinching surfaces 27, 28 which permit the retention of the body between, for example, two adjacent fingers. Indeed, in the embodiment shown, the opposing clinching surfaces are molded into, and form a part of, the inner cavity. It is contemplated that the clinching surfaces may comprise rubber or neoprene members which are co-molded, adhered or otherwise associated with inner cavity 23.

In other embodiments, such as the embodiment shown in FIG. 2, the grasping assembly may comprise a hoop-like member, such as ring grasping member 30. In such an embodiment, the body is retained by extending a finger through ring grasping member 30. Advantageously, such a configurations allows the wearer to rotate the device to a position of operation (wherein the device is positioned proximate the palm of the hand) to a position of storage or retention (wherein the device is positioned proximate the outer hand).

In yet another embodiment, shown in FIG. 3, gripping assembly 24 may comprise a dual ring grasping configuration comprising ring grasping members 30, 32. In such an embodiment, the pair of ring grasping members provide additional control, as they facilitate retention by a plurality of fingers.

Body adhesion member 26 may comprise an adhesive or a tape member which can be applied to each of the stethoscope apparatus and to the skin of the patient to retain the device in position. It is contemplated that such a body adhesion member may comprise a tape which attaches to inner cavity 23 or an adhesive which is applied to body interfacing surface 22. Indeed, any number of different solutions are contemplated which facilitate the retention of the stethoscope in a desired orientation relative to the user. Advantageously, such a configuration facilitates the retention of the stethoscope in a desired orientation while the user is free to undertake other tasks.

Sensor assembly 14 is shown in FIG. 1 as comprising at least one sensor, such as sensor 36, and control assembly 38. Sensor 36 is positioned proximate body interfacing surface 22 (and in certain embodiments may be integrated therewith). The sensor may comprise any number of different sensors, including, but not limited to pressure sensors, movement sensors, a microphone, among others. Such sensors are capable of sensing the beating heart as it is transmitted through body interfacing surface 22, and converting same into an electrical (either analog or digital) signal.

The sensor output is associated with control assembly 38. Control assembly comprises electronic circuitry having at least one input (associated with the sensor), at least one output (associated with output indicator devices 16), and computing circuitry. In certain embodiments, the control assembly may comprise a relatively simple analog circuit. In other assemblies, the control assembly may comprise a substantially sophisticated digital circuit. The control assembly may further include a battery for powering each of the sensor assembly, the output indicator devices, storage devices and/or remote communication assembly. The battery may be rechargeable or replaceable. In one embodiment, the battery may include an indicator (or may interface with the visual output indicator devices) to indicate a low battery condition. A plug may be provided which can be coupled to a transformer or other device intended for charging.

The control assembly may further include a clock or other timing device which is capable of recording time (i.e., clock) or recording the passage of time (i.e., stopwatch). The clock may have a display associated with body 12.

Output indicator devices 16 are associated with control assembly 38, and are directed by control assembly 38 to reflect information transmitted to the control assembly from the sensor 36. For example, the output indicator devices may comprise one or more of various visual, audio and tactile output devices 40, 44, 46, respectively. Among others, visual output 40 comprises any one or more of an LED, a LCD and/or an incandescent lamp. Among others, audio output 44 may comprise a pezio electric element or a speaker capable of audio sounds. Tactile output may comprise any number of different vibration inducers.

Storage device 18 is shown in FIG. 1 as comprising non-volatile flash memory, or other RAM which permits the storage of data as received from sensor 36. For example, the storage device can be associated with the control assembly. In other embodiments, storage device 18 may comprise a removable, flash type memory, including but not limited to CF, SM, MS, MMC, SD, among others. The storage device can be used to store data pertaining to the heart beat of a user over time.

Remote communication assembly 20 is shown in FIG. 1 as comprising a connection to a remote device. In one embodiment, the remote connection assembly may comprise a mini plug which is capable of receiving the jack of headphones. In other embodiments, the remote communication assembly may comprise an IR port, or an RF communication device, which facilitates the communication with a remote device. In another embodiment, the remote communication assembly may comprise a Bluetooth communication device. Advantageously, the remote communication assembly permits placement of body 10 in a remote location (i.e., not visible, or precarious position), yet permits the use thereof, and the receipt of information therefrom. One type of remote connection may comprise a remotely positioned output indicator (audio/visual/tactile), or a remote data gathering device (i.e., PDA or computer).

Advantageously, the device permits monitoring by observing blinking lights or audible signals that beep (i.e., without having a device attached by way of a cord to a user). Moreover, it is possible to adhere the device to the patient so as to facilitate multi-tasking by a user. Furthermore, the device can facilitate use even in greatly confined and dangerous areas. In particular, after attachment, the user can move away from the patient, while still monitoring the patient (useful in, for example, a car accident). Furthermore, the device can be used in place of electrodes, which generally present complications with the vast number of different wires and positioned devices.

Advantageously, the device can be packaged in a sterile sealed container. As such, when needed, the user can break the sealed container, with the understanding that the device is properly sterilized. In certain embodiments, the stethoscope can be re sterilized or, in other embodiments, it may be disposable.

It is contemplated that an emergency unit may include a number of the stethoscopes, such that in the event of an emergency, a number of the stethoscopes can be in use simultaneously. It is contemplated that the devices may each include a unique identifier so that any remote data gathering equipment can discern between the various devices. It is likewise contemplated that each first aid kit may be configured to include such a device.

The foregoing description merely explains and illustrates the invention and the invention is not limited thereto except insofar as the appended claims are so limited, as those skilled in the art who have the disclosure before them will be able to make modifications without departing the scope of the invention.

1. A stethoscope apparatus comprising:
a body having a body interfacing surface;
a sensor assembly including a sensor associated with the body interfacing surface; and
an output indicator device coupled with the sensor assembly so as to provide at least one of a visual, audio and tactile indicators of the condition of the sensor.

2. The apparatus of claim 1 further comprising a storage device capable of storing data pertaining to the sensor.

3. The apparatus of claim 1 further comprising a remote communication assembly facilitating the transfer of data or sensor condition to a remote receiver or device.
